(19) United States
(12) Patent Application Publication
(10) Pub. No.: US 2001/0025099 A1
(43) Pub. Date: Sep. 27, 2001

(54) G protein-coupled receptor polypeptides and polyncleotides

(76) Inventors: Nabil Elshourbagy, West Chester, PA (US); Lisa Vawter, West Chester, PA (US)

Correspondence Address: Ratner & Prestia, P.O. Box 980, Valley Forge, PA, 19482-0980, US

(21) Appl. No.: 09/826,508

(22) Filed: Apr. 5, 2001

Related U.S. Application Data
(63) Continuation of application No. 09/075,468, filed on May 8, 1998.
(63) Continuation of application No. 09/274,080, filed on Mar. 22, 1999.
(63) Continuation of application No. 09/363,203, filed on Jul. 29, 1999.
(63) Continuation of application No. 09/384,610, filed on Aug. 27, 1999.
(63) Continuation of application No. 09/144,779, filed on Sep. 1, 1998.
(63) Continuation of application No. 09/075,464, filed on May 8, 1998.
(63) Continuation of application No. 09/193,212, filed on Nov. 17, 1998.
(63) Continuation of application No. 09/337,105, filed on Jun. 21, 1999, now abandoned.
(63) Continuation of application No. 09/188,837, filed on Nov. 9, 1998.
(63) Continuation of application No. 09/253,216, filed on Feb. 19, 1999.
(63) Continuation of application No. 09/287,034, filed on Apr. 6, 1999.
(63) Continuation of application No. 09/425,406, filed on Oct. 22, 1999.
(63) Continuation of application No. 09/260,360, filed on Mar. 1, 1999.
(63) Continuation of application No. 09/328,603, filed on Jun. 9, 1999.
(60) Provisional application No. 60/075,307, filed on Feb. 20, 1998.

(51) Int. Cl. C07H 21/04; C07K 14/705; C12N 5/06

(52) U.S. Cl. 536/23.1; 530/350; 435/325; 435/69.1

(57) ABSTRACT

Polypeptides and polynucleotides and methods for producing such polypeptides by recombinant techniques are disclosed. Also disclosed are methods for utilizing polypeptides and polynucleotides in therapy, and diagnostic assays for such.

This application is a continuation of co-pending application Ser. Nos. 09/075,468, filed May 8, 1998, 09/274,080 filed Mar. 22, 1999, 09/363,203 filed Jul. 29, 1999, 09/384,610 filed Aug. 27, 1999, 09/144,779 filed Sep. 1, 1998, 09/075,464 filed May 8, 1998, 09/193,212 filed Nov. 17, 1998, 09/337,105 filed Jun. 21, 1999, 09/188,837 filed Nov. 9, 1998, 09/253,216 filed Feb. 19, 1999, 09/287,034 filed Apr. 6, 1999, 09/425,406 filed Oct. 22, 1999, 09/260,360, filed Mar. 1, 1999, and 09/328,603, filed Jun. 9, 1999.

Application Ser. No. 09/075,468 claims priority to U.S. Provisional Application No. 60/075,307, filed Feb. 20, 1998. Application Ser. No. 09/274,080 is a divisional of a U.S. application Ser. No. 08/958,240, filed Oct. 27, 1997 which in turn claims priority to U.S. Provisional Application No. 60/050,122, filed Jun. 18, 1997. Application Ser. No. 09/188,837 is a division of application Ser. No. 08/827,291, filed Mar. 28, 1997. Application Ser. No. 09/253,216 is a Continuation-in-Part application of U.S. application Ser. No. 09/183,253, filed Oct. 30, 1998, which in turn claims the benefit of priority of U.S. Provisional Application No. 60/075,306, filed Feb. 20, 1998, and of U.K. Application No. 9817907.0, filed Aug. 17, 1998. Application Ser. No. 09/287,034 claims the benefit of U.K. Application No. 9807722.5, filed Apr. 8, 1998. Application Ser. No. 09/425,406 claims the benefit of U.S. application Ser. No. 09/247,111, filed Feb. 9, 1999 and U.K. Application No. 9825423.8, filed Nov. 19, 1998. Application Ser. No. 09/260,360 is a divisional of application Ser. No. 08/775,428, filed Jan. 9, 1997. Lastly, application Ser. No. 09/328,603 is a divisional of application Ser. No. 08/955,713, filed Oct. 23, 1997, which in turn claims the benefit of U.S. Provisional Application No. 60/050,124, filed Jun. 18, 1997.

### The entire contents of each of the foregoing applications are incorporated herein by reference.

### FIELD OF THE INVENTION

This invention relates to newly identified polypeptides and polynucleotides encoding such polypeptides, to their use in therapy and in identifying compounds which may be agonists, antagonists and/or inhibitors which are potentially useful in therapy, and to production of such polypeptides and polynucleotides.

### BACKGROUND OF THE INVENTION

The drug discovery process is currently undergoing a fundamental revolution as it embraces ‘functional genomics’, that is, high throughput genome- or gene-based biology. This approach is rapidly superseding earlier approaches based on ‘positional cloning’. A phenotype, that is, a biological function or genetic disease, would be identified and this would then be tracked back to the responsible gene, based on its genetic map position.

Functional genomics relies heavily on the various tools of bioinformatics to identify gene sequences of potential interest from the many molecular biology databases now available. There is a continuing need to identify and characterize further genes and their related polypeptides/proteins, as targets for drug discovery.

It is well established that many medically significant biological processes are mediated by proteins participating in signal transduction pathways that involve G-proteins and/or second messengers, e.g., cAMP (Lefkowitz, Nature, 1991, 351:353-354). These proteins maybe referred to as proteins participating in pathways with G-proteins or PPG proteins. Some examples of these proteins include the GPC receptors, such as those for adrenergic agents and dopamine (Kobilka, B. K., et al., Proc. Natl Acad. Sci., USA, 1987, 84:46-50; Kobilka, B. K., et al., Science, 1987, 238:650-656; Bunzow, J. R., et al., Nature, 1988, 336:783-787), G-proteins themselves, effector proteins, such as phospholipase C, adenyl cyclase, and phosphodiesterase, and actuator proteins, such as protein kinase A and protein kinase C (Simon, M. I., et al., Science, 1991, 252:802-8).

For example, in one form of signal transduction, the effect of hormone binding is activation of the enzyme, adenylate cyclase, inside the cell. Enzyme activation by hormones is dependent on the presence of the nucleotide GTP. GTP also influences hormone binding. A G-protein connects the hormone receptor to adenylate cyclase, exchanging GTP for bound GDP when activated by a hormone receptor. The GTP-carrying form then binds to activated adenylate cyclase. Hydrolysis of GTP to GDP, catalyzed by the G-protein itself, returns the G-protein to its basal, inactive form. Thus, the G-protein serves a dual role, as an intermediate that relays the signal from receptor to effector, and as a clock that controls the duration of the signal.

The membrane protein gene superfamily of G-protein coupled receptors has been characterized as having seven putative transmembrane domains. The domains are believed to represent transmembrane α-helices connected by extracellular or cytoplasmic loops. G-protein coupled receptors include a wide range of biologically active receptors, such as hormone, viral, growth factor and neuroreceptors.

G-protein coupled receptors (otherwise known as 7TM receptors) have been characterized as including these seven conserved hydrophobic stretches of about 20 to 30 amino acids, connecting at least eight divergent hydrophilic loops. The G-protein family of coupled receptors includes dopamine receptors which bind to neuroleptic drugs used for treating psychotic and neurological disorders. Other examples of members of this family include, but are not limited to, calcitonin, adrenergic, endothelin, cAMP, adenosine, muscarinic, acetylcholine, serotonin, histamine, thrombin, kinin, follicle stimulating hormone, opsins, endothelial differentiation gene-1, rhodopsins, odorant, and cytomegalovirus receptors.

Most G-protein coupled receptors have single conserved cysteine residues in each of the first two extracellular loops which form disulfide bonds that are believed to stabilize functional protein structure. The 7 transmembrane regions are designated as TM1, TM2, TM3, TM4, TM5, TM6, and TM7. TM3 has been implicated in signal transduction.

Phosphorylation and lipidation (palmitylation or farnesylation) of cysteine residues can influence signal transduction of some G-protein coupled receptors. Most G-protein coupled receptors contain potential phosphorylation sites within the third cytoplasmic loop and/or the carboxy terminus. For several G-protein coupled receptors, such as the β-adrenoreceptor, phosphorylation by protein kinase A and/or specific receptor kinases mediates receptor desensitization.

For some receptors, the ligand binding sites of G-protein coupled receptors comprise hydrophilic sockets formed by several G-protein coupled receptor transmembrane domains, said socket being surrounded by hydrophobic residues of the G-protein coupled receptors. The hydrophilic side of each G-protein coupled receptor transmembrane helix is postulated to face inward and form polar ligand binding site. TM3 has been implicated in several G-protein coupled receptors as having a ligand binding site, such as the TM3 aspartate residue. TM5 serines, a TM6 asparagine and TM6 or TM7 phenylalanines or tyrosines are also implicated in ligand binding.

G-protein coupled receptors can be intracellularly coupled by heterotrimeric G-proteins to various intracellular enzymes, ion channels and transporters (see, Johnson et al., Endoc. Rev., 1989, 10:317-331) Different G-protein α-subunits preferentially stimulate particular effectors to modulate various biological functions in a cell. Phosphorylation of cytoplasmic residues of G-protein coupled receptors have been identified as an important mechanism for the regulation of G-protein coupling of some G-protein coupled receptors. G-protein coupled receptors are found in numerous sites within a mammalian host.

Over the past 15 years, nearly 350 therapeutic agents targeting 7 transmembrane (7 TM) receptors have been successfully introduced onto the market.

### SUMMARY OF THE INVENTION

The present invention relates to polypeptides and polynucleotides, recombinant materials and methods for their production. In another aspect, the invention relates to methods for using such polypeptides and polynucleotides, including the treatment of any or several of a wide variety of diseases, including infections such as bacterial, fungal, protozoan and viral infections, particularly infections caused by HIV-1 or HIV-2; pain; cancers; diabetes, obesity; anorexia; bulimia; asthma; Parkinson's disease; acute heart failure; hypotension; hypertension; urinary retention; osteoporosis; angina pectoris; myocardial infarction; stroke; ulcers; asthma; allergies; benign prostatic hypertrophy; migraine; vomiting; psychotic and neurological disorders, including anxiety, schizophrenia, manic depression, depression, delirium, dementia, and severe mental retardation; and dyskinesias, such as Huntington's disease or Gilles dela Tourett's syndrome, hereinafter referred to as “the Diseases”, amongst others. In a further aspect, the invention relates to methods for identifying agonists and antagonists/inhibitors using the materials provided by the invention, and treating conditions associated with an imbalance of polynucleotides and polypeptides with the identified compounds. In a still further aspect, the invention relates to diagnostic assays for detecting diseases associated with inappropriate polypeptide and polynucleotide activity or levels.

### DESCRIPTION OF THE INVENTION

In a first aspect, the present invention relates to polypeptides. Such peptides include isolated polypeptides comprising an amino acid sequence which has at least 70% identity, preferably at least 80% identity, more preferably at least 90% identity, yet more preferably at least 95% identity, most preferably at least 97-99% identity, to that of the polypeptide sequence over the entire length of the polypeptide sequence. Such polypeptides include those comprising the amino acids described in Table 1, and more specifically the amino acid sequence set forth in any one of SEQ ID NOS: 2, 4, 6, 8, 10, 12, 14, 16, 20, 22, 24, 26, 28, 30, 32, 34 or 36.

Further, peptides of the present invention include isolated polypeptides in which the amino acid sequence has at least 70% identity, preferably at least 80% identity, more preferably at least 90% identity, yet more preferably at least 95% identity, most preferably at least 97-99% identity, to the an amino acid sequence identified above over the entire length of the amino acid sequence.

Further, peptides of the present invention include isolated polypeptides encoded by a polynucleotide comprising the nucleotide sequence set forth in any one of SEQ ID NOS: 1, 3, 5, 7, 9, 11, 13, 15, 17, 18, 19, 21, 23, 25, 27, 29, 31, 33, or 35 and described in more detail below.

Polypeptides of the present invention are members of the seven transmembrane receptor family of polypeptides. They are therefore of interest because G protein-coupled receptors, more than any other gene family, are targets of successful pharmaceutical intervention. These properties are hereinafter referred to as “activity” or “polypeptide activity” or “biological activity”. Also included amongst these activities are antigenic and immunogenic activities of the polypeptides, in particular the antigenic and immunogenic activities of the polypeptides having the amino acid sequences as set forth in the sequence listing. Preferably, a polypeptide of the present invention exhibits at least one biological activity.

The polypeptides of the present invention may be in the form of the “mature” protein or may be a part of a larger protein such as a fusion protein. It is often advantageous to include an additional amino acid sequence which contains secretory or leader sequences, pro-sequences, sequences which aid in purification such as multiple histidine residues, or an additional sequence for stability during recombinant production.

The present invention also includes variants of the aforementioned polypeptides, that is polypeptides that vary from the referents by conservative amino acid substitutions, whereby a residue is substituted by another with like characteristics. Typical substitutions are among Ala, Val, Leu and Ile; among Ser and Thr; among the acidic residues Asp and Glu; among Asn and Gln; and among the basic residues Lys and Arg; or aromatic residues Phe and Tyr. Particularly preferred are variants in which several, 5-10, 1-5, 1-3, 1-2 or 1 amino acids are substituted, deleted, or added in any combination.

Polypeptides of the present invention can be prepared in any suitable manner. Such polypeptides include isolated naturally occurring polypeptides, recombinantly produced polypeptides, synthetically produced polypeptides, or polypeptides produced by a combination of these methods. Means for preparing such polypeptides are well understood in the art.

In a further aspect, the present invention relates to polynucleotides. Such polynucleotides include isolated polynucleotides comprising a nucleotide sequence encoding a corresponding polypeptide which has at least 70% identity, preferably at least 80% identity, more preferably at least 90% identity, yet more preferably at least 95% identity, to the corresponding amino acid sequence identified in Table 1, over the entire length of amino acid sequence. In this regard, polypeptides which have at least 97% identity are highly preferred, whilst those with at least 98-99% identity are more highly preferred, and those with at least 99% identity are most highly preferred. For example, such polynucleotides include a polynucleotide comprising the nucleotide sequence of SEQ ID NO:1 encoding the polypeptide of SEQ ID NO:2, etc., as shown in Table 1.

Further polynucleotides of the present invention include isolated polynucleotides comprising a nucleotide sequence that has at least 70% identity, preferably at least 80% identity, more preferably at least 90% identity, yet more preferably at least 95% identity, to a nucleotide sequence encoding a polypeptide set forth in Table 1, over the entire coding region. In this regard, polynucleotides which have at least 97% identity are highly preferred, whilst those with at least 98-99% identity are more highly preferred, and those with at least 99% identity are most highly preferred.

Further polynucleotides of the present invention include isolated polynucleotides comprising a nucleotide sequence which has at least 70% identity, preferably at least 80% identity, more preferably at least 90% identity, yet more preferably at least 95% identity, to the polynucleotide sequences set forth in Table 1, over the entire length of sequence. In this regard, polynucleotides which have at least 97% identity are highly preferred, whilst those with at least 98-99% identity are more highly preferred, and those with at least 99% identity are most highly preferred. Such polynucleotides include a polynucleotide comprising the nucleotide sequence of any one of SEQ ID NOS: 1, 3, 5, 7, 9, 11, 13, 15, 17, 18, 19, 21, 23, 25, 27, 29, 31, 33, or 35.

The invention also provides polynucleotides which are complementary to all the above described polynucleotides.

Table 1 below provides the polynucleotide sequence identification numbers and polypeptide sequence identification numbers of each family member and the respective data for each member.

TABLE 1

| Polynucleotide | Polypeptide | |
|---|---|---|
| SEQ ID NO: | SEQ ID NO: | Detailed Description of the Polynucleotide and Polyeptide |
| 1 | 2 | Gene Name: Mucilage |
| | | The nucleotide sequence of SEQ ID NO:1 shows homology |
| | | with genembl:u80895 (Mus musculus CAG trinucleotide repeat; |
| | | Kim, S. J., Shon, B. H., Kang, J. H., Hahm, K. -S., Yoo, O. J., Park, Y. S. |
| | | and Lee, K. -K. Biochem. Biophys. Res. Commun. 240, 239-243 |
| | | (1997). The nucleotide sequence of SEQ ID NO:1 is a cDNA |
| | | sequence and comprises a polypeptide encoding sequence |
| | | (nucleotide 150 to 1592) encoding a polypeptide of 481 amino |
| | | acids, the polypeptide of SEQ ID NO:2. |
| | | The polypeptide of the SEQ ID NO:2 is structurally related |
| | | to other proteins of the seven transmemebrance receptor family, |
| | | having homology and/or structural similarity with nonred:2388706 |
| | | putative GPCR (Donohue, P. J., Shapira, H., Mantey, S. A., |
| | | Hampton, L. L., Jensen, R. T. and Battey, J. F., submitted). |
| 3 | 4 | The nucleotide sequence of SEQ ID NO:3 and the peptide |
| | | sequence encoded thereby are derived from EST (Expressed |
| | | Sequence Tag) sequences. It is recognized by those skilled in the |
| | | art that there will inevitably be some nucleotide sequence reading |
| | | errors in EST sequences (see Adams, M. D. et al, Nature 377 (supp) |
| | | 3, 1995). Accordingly, the nucleotide sequence of SEQ ID NO:3 |
| | | and the peptide sequence encoded therefrom are therefore subject |
| | | to the same inherent limitations in sequence accuracy. |
| | | Furthermore, the peptide sequence encoded by SEQ ID NO:3 |
| | | comprises a region of identity or close homology and/or close |
| | | structural similarity (for example a conservative amino acid |
| | | difference) with the closest homologous or structurally similar |
| | | protein. |
| | | Polynucleotides of this invention may be obtained, using |
| | | standard cloning and screening techniques, from a cDNA library |
| | | derived from mRNA in cells of human brain, using the expressed |
| | | sequence tag (EST) analysis. |
| 5 | 6 | Gene Name: H7TBA62 |
| | | The cDNA sequence of SEQ ID NO:5 contains an open |
| | | reading frame (nucleotide number 1020 to 2141) encoding a |
| | | polypeptide of 374 amino acids of SEQ ID NO:6. SEQ ID NO:6 |
| | | has about 32% identity (using FASTA) in 300 amino acid residues |
| | | with Human Somatostatin Receptor Type 4 (PNAS 90:4196-4200, |
| | | 1993). Furthermore, H7TBA62 (SEQ ID NO:2) is 27% identical |
| | | to the Human RDC-1 homolog Receptor over 318 amino acid |
| | | residues (PNAS 88:4986-4990, 1991). SEQ ID NO:5 has about |
| | | 55% identity (using FASTA) in 1079 nucleotide residues with |
| | | Human Somatostatin Receptor Type 3 (FEBS Lett. 321, 279-284, |
| | | 1993). Furthermore, H7TBA62 (SEQ ID NO:5) is 56% identical |
| | | to Human APJ Receptor over 596 nucleotide base residues (Gene |
| | | 136, 355-360, 1993). |
| | | Further embodiments are polynucleotides encoding |
| | | H7TBA62 variants comprising the amino acid sequence of |
| | | H7TBA62 SEQ ID NO:6 in which several, 5-10, 1-5, 1-3, 1-2 or 1 |
| 7 | 8 | amino acid residues are substituted, deleted or added, in any |
| | | combination. |
| | | Among the polynucleotides of the present invention is the |
| | | sequence set forth in SEQ ID NO:7, encoding the amino acid |
| | | sequence of SEQ ID NO:8. |
| | | One polynucleotide of this invention encoding H7TBA62 |
| | | may be obtained using standard cloning and screening, from a |
| | | cDNA library derived from mRNA in cells of human brain using |
| | | the expressed sequence tag (EST) analysis. |
| 9 | 10 | Gene Name: Octoray |
| | | The polynucleotide sequence of SEQ ID NO:9 shows |
| | | homology with KIAA0001 (Nomura N., Miyajima N., Sazuka T, |
| | | Tanaka A, Kawarabayashi Y, Sato S, Nagase T, Seki N, Ishikawa |
| | | K, Tabata S 1994. DNA Res. 1, 27-35). The polynucleotide |
| | | sequence of SEQ ID NO:9 is a cDNA sequence that encodes the |
| | | polypeptide of SEQ ID NO:10. The polynucleotide sequence |
| | | encoding the polypeptide of SEQ ID NO:10 may be identical to the |
| | | polypeptide encoding sequence of SEQ ID NO:9 or it may be a |
| | | sequence other than SEQ ID NO:9, which, as a result of the |
| | | redundancy (degeneracy) of the genetic code, also encodes the |
| | | polypeptide of SEQ ID NO:10. |
| | | The polypeptide of the SEQ ID NO:10 is related to other |
| | | proteins of the this family, having homology and/or structural |
| | | similarity with KIAA0001 (Normura N, Miyajima N, Sazuka T, |
| | | Tanaka A, Kawarabayashi Y, Sato S, Nagase T, Seki N, Ishikawa |
| | | K, Tabata S 1994. DNA Res. 1, 47-56). |
| | | Polynucleotides of the present invention may be obtained |
| | | using standard cloning and screening techniques from a cDNA |
| | | library derived from mRNA in cells of human placenta and testis. |
| 11 | 12 | Gene Name: GPCR, TheAnt |
| | | The polynucleotide sequence of SEQ ID NO:11 shows |
| | | homology with human mas oncogene (Young D., et al., Cell 1986. |
| | | 45:711-718). The polynucleotide sequence of SEQ ID NO:11 is a |
| | | cDNA sequence that encodes the polypeptide of SEQ ID NO:12. |
| | | The polynucleotide sequence encoding the polypeptide of SEQ ID |
| | | NO:12 may be identical to the polypeptide encoding sequence of |
| | | SEQ ID NO:11 or it may be a sequence other than SEQ ID NO:11, |
| | | which, as a result of the redundancy (degeneracy) of the genetic |
| | | code, also encodes the polypeptide of SEQ ID NO:12. The |
| | | polypeptide of SEQ ID NO:12 is related to other proteins of the G- |
| | | Protein Coupled family, having homology and/or structural |
| | | similarity with human mas oncogene (Young D., et al., Cell 1986. |
| | | 45:711-718). |
| | | Polynucleotides of this invention may be obtained using |
| | | standard cloning and screening techniques from a cDNA library |
| | | derived from mRNA in cells of human ovary and spleen. |
| 13 | 14 | Gene Name: GABAB1AA |
| | | The nucleotide sequence of SEQ ID NO:13 shows |
| | | homology with GABAB1a. The nucleotide sequence of SEQ ID |
| | | NO:13 is a cDNA sequence and comprises a polypeptide encoding |
| | | sequence (nucleotide 1 to 2880) encoding a polypeptide of 960 |
| | | amino acids, the polypeptide of SEQ ID NO:14. The nucleotide |
| | | sequence encoding the polypeptide of SEQ ID NO:14 may be |
| | | identical to the polypeptide encoding sequence contained in SEQ |
| | | ID NO:13 or it may be a sequence other than the one contained in |
| | | SEQ ID NO:13, which, as a result of the redundancy (degeneracy) |
| | | of the genetic code, also encodes the polypeptide of SEQ ID |
| | | NO:14. The polypeptide of SEQ ID NO:14 is structurally related |
| | | to other proteins of the GABAB family, having homology and/or |
| | | structural similarity with GABAB1a. |
| | | Polynucleotides of the present invention may be obtained, |
| | | using standard cloning and screening techniques, from a cDNA |
| | | library derived from mRNA in cells of human brain and human |
| | | fetal brain, using the expressed sequence tag (EST) analysis. |
| 15 | 16 | Gene Name: GPRW |
| | | The nucleotide sequence of SEQ ID NO:15 shows |
| | | homology with GPCRW receptor: Gantz, I., Muraoka, A., |
| | | Yang, Y. K., Samuelson, L. C., Zimmerman, E. M., Cook, H. and |
| | | Yamada, T. Genomics 42 (3), 462-466 (1997). The nucleotide |
| | | sequence of SEQ ID NO:15 is a cDNA sequence and comprises a |
| | | polypeptide encoding sequence (nucleotide 1 to 993) encoding a |
| | | polypeptide of 331 amino acids, the polypeptide of SEQ ID |
| | | NO:16. The nucleotide sequence encoding the polypeptide of SEQ |
| | | ID NO:16 maybe identical to the polypeptide encoding sequence |
| | | contained in SEQ ID NO:15 or it may be a sequence other than the |
| | | one contained in SEQ ID NO:15, which, as a result of the |
| | | redundancy (degeneracy) of the genetic code, also encodes the |
| | | polypeptide of SEQ ID NO:16. The polypeptide of SEQ ID NO:16 |
| | | is structurally related to other proteins of the Purinergic receptor |
| | | family, having homology and/or structural similarity with GPCRW |
| | | receptor, Gantz, I., Muraoka, A., Yang, Y. K., Samuelson, L. C., |
| | | Zimmerman, E. M., Cook, H. and Yamada, T. Genomics 42 (3), 462- |
| | | 466 (1997). |
| | | Polynucleotides of this invention may be obtained, using |
| | | standard cloning and screening techniques, from a cDNA library |
| | | derived from mRNA in cells of human placenta, using the |
| | | expressed sequence tag (EST) analysis. |
| 17 | 18 | The published GPCRW was identified from the public |
| | | database as a potential 7TM receptor. Oligonucleotides (5′) were |
| | | designed at the 5′ and the 3′ end of the clone. The 5′ primer was |
| | | (GAGCTATTTTAACAGAAGCAACTC) (SEQ ID NO:17) and |
| | | the 3′ primer was (AGGGACTTGATAGTATTATACAG) (SEQ |
| | | ID NO:18). These oligonucleotides were used to PCR a 1 kb |
| | | fragment using the human placenta cDNA as a template. The PCR |
| | | fragment was subcloned into pCR2.1 vector and sequenced. |
| | | Comparison of the nucleotide sequence of the GPRW (SEQ ID |
| | | NO:15) with the published GPCRW revealed 4 amino acid |
| | | differences. The cloning procedure was performed twice to |
| | | confirm the changes in the amino acid sequences. |
| | | The gene of this invention maps to human chromosome |
| | | 13q32 |
| 19 | 20 | Gene Name: Mouse KIAA0001 |
| | | The nucleotide sequence of SEQ ID NO:19 shows |
| | | homology with Human KIAA0001, (Monura N., DNA Res |
| | | 1994;1(1): 27-35). The nucleotide sequence of SEQ ID NO:19 is a |
| | | cDNA sequence and comprises a polypeptide encoding sequence |
| | | (nucleotide 1 to 1017) encoding a polypeptide of 338 amino acids, |
| | | the polypeptide of SEQ ID NO:20. The nucleotide sequence |
| | | encoding the polypeptide of SEQ ID NO:20 may be identical to the |
| | | polypeptide encoding sequence contained in SEQ ID NO:19 or it |
| | | may be a sequence other than the one contained in SEQ ID NO:19, |
| | | which, as a result of the redundancy (degeneracy) of the genetic |
| | | code, also encodes the polypeptide of SEQ ID NO:20. |
| | | The polypeptide of SEQ ID NO:20 is structurally related to |
| | | other proteins of the 7TM receptor family, having homology and/or |
| | | structural similarity with Human KIAA0001. |
| | | These polynucleotides may be obtained, using standard |
| | | cloning and screening techniques, from a cDNA library derived |
| | | from mRNA in cells of mouse kidney “six weeks,” using the |
| | | expressed sequence tag (EST) analysis. |
| 21 | 22 | Gene Name: AXOR16 |
| | | The polynucleotide sequence of SEQ ID NO:21 shows |
| | | homology with Gadus morhua neuropeptide (NPYRB) F. The |
| | | polynucleotide sequence of SEQ ID NO:21 is a cDNA sequence |
| | | that encodes the polypeptide of SEQ ID NO:22. The |
| | | polynucleotide sequence encoding the polypeptide of SEQ ID |
| | | NO:22 may be identical to the polypeptide encoding sequence of |
| | | SEQ ID NO:21 or it may be a sequence other than SEQ ID NO:21, |
| | | which, as a result of the redundancy (degeneracy) of the genetic |
| | | code, also encodes the polypeptide of SEQ ID NO:22. The |
| | | polypeptide of SEQ ID NO:22 is related to other proteins of the G |
| | | protein coupled receptor 7TM family, having homology and/or |
| | | structural similarity with Danio rerio neuropeptide Y (NPYRYA) |
| | | [P. Starback et. al. DNA Cell Biology 16(11), 1357-1363, 1997]. |
| | | Polynucleotides of this invention may be obtained using |
| | | standard cloning and screening techniques from a cDNA library |
| | | derived from mRNA in cells of human kidney and testis. |
| | | The gene of this invention maps to human chromosome |
| | | 11q12.2. |
| 23 | 24 | Gene Name: OLRCC15 |
| | | The receptor of the invention is structurally related to other |
| | | proteins of the olfactory receptor, as shown by the results of |
| | | sequencing the cDNA encoding human OLRCC15 receptor. The |
| | | cDNA sequence contains an open reading frame encoding a |
| | | polypeptide of 316 amino acids. Amino acid sequence of SEQ ID |
| | | NO:24 has about 44.7% identity (using TFASTA) in 304 amino |
| | | acid residues with odorant receptor (G. Drutel, J. M. Arrang, J. |
| | | Diaz, C. Wisnewsky, K. Schwartz & J. C. Schwartz (1995), |
| | | Cloning of OL1, a putative olfactory receptor and its expression in |
| | | the developing rat heart, Recept. Channels 3, 33-40). |
| | | Furthermore, the amino acid sequence of OLRCC15 (SEQ ID |
| | | NO:24) is 41.0% identical to mouse G-protein coupled receptor, |
| | | olfactory receptor over 312 amino acid residues (P. Nef, I. |
| | | Hermans-Borgmeyer, H. Artieres-Pin, L. L. Beasley, V. E. Dionne |
| | | & S. F. Heinemann (1992), Spatial pattern of receptor expression in |
| | | the olfactory epithelium, Proc. Natl. Acad. Sci. U.S.A. 89: 8948- |
| | | 8952). Nucleotide sequence, SEQ ID NO:23, has about 67.82% |
| | | identity (using BlastN) in 463 nucleotide residues with H. sapiens |
| | | mRNA for TPCR100 protein (Vanderhaeghen, P., Schurmans, S., |
| | | Vassart, G. and Parmentier, M. Male germ cells from several |
| | | mammalian species express a specific repertoire of olfactory |
| | | receptor genes. GeneBank ACCESSION X89666. Submitted (12- |
| | | JUL-1995) P. Vanderhaeghen, Universit Libre de Bruxelles, |
| | | IRIBHN, ULB Campus Erasme, 808 route de Lennik, 1070 |
| | | Bruxelles, BELGIUM). Furthermore, OLRCC15 (SEQ ID NO:23) |
| | | is 55.94% identical to H. sapiens HGMP07I gene for olfactory |
| | | receptor over 935 nucleotide base residues (M. Parmentier, F. |
| | | Libert, S. Schurmans, S. Schiffmann, A. Lefort, D. Eggerickx, C. |
| | | Ledent, C. Mollereau, C. Gerard, J. Perret, J. A. Grootegoed & G. |
| | | Vassart (1992), Expression of members of the putative olfactory |
| | | receptor gene family in mammalian germ cells, Nature 355, 453- |
| | | 455). |
| | | One polynucleotide of the present invention encoding |
| | | OLRCC15 receptor may be obtained using standard cloning and |
| | | screening, from a cDNA library derived from mRNA in cells of |
| | | human colon and human blood using the expressed sequence tag |
| | | (EST) analysis. |
| 25 | 26 | Gene Name: GABAB-R2a |
| | | The nucleotide sequence of SEQ ID NO:25 shows |
| | | homology with GABAB-R2, K. Jones et al (1998). Nature, 396, |
| | | 674-679. J. White et al (1998) Nature, 396, 679-682. K. Kaupmann |
| | | et al (1998) Nature 379, 683-686.. The nucleotide sequence of |
| | | SEQ ID NO:25 is a cDNA sequence and comprises a polypeptide |
| | | encoding sequence (nucleotide 31 to 2652) encoding a polypeptide |
| | | of 874 amino acids, the polypeptide of SEQ ID NO:26. The |
| | | nucleotide sequence encoding the polypeptide of SEQ ID NO:26 |
| | | may be identical to the polypeptide encoding sequence contained |
| | | in SEQ ID NO:25 or it may be a sequence other than the one |
| | | contained in SEQ ID NO:25, which, as a result of the redundancy |
| | | (degeneracy) of the genetic code, also encodes the polypeptide of |
| | | SEQ ID NO:26. The polypeptide of SEQ ID NO:26 is structurally |
| | | related to other proteins of the G-protein coupled receptor family, |
| | | having homology and/or structural similarity with GABAB-R2, (K. |
| | | Jones, et al., Nature, 396, 674-679 (1998); J. White et al., Nature, |
| | | 396, 679-682 (1998); and K. Kaupmann et al., Nature 379, 683- |
| | | 686 (1998)). |
| 27 | 28 | The nucleotide sequence of SEQ ID NO:27 and the peptide |
| | | sequence encoded thereby (SEQ ID NO:28) are derived from EST |
| | | (Expressed Sequence Tag) sequences. |
| | | Polynucleotides of the present invention may be obtained, |
| | | using standard cloning and screening techniques, from a cDNA |
| | | library derived from mRNA in cells of human hippocampus, using |
| | | the expressed sequence tag (EST) analysis. |
| 29 | 30 | Gene Name: AXOR3 |
| | | The nucleotide sequence of SEQ ID NO:29 shows |
| | | homology with Human putative G-protein coupled receptor RAIG1 |
| | | (Y. Cheng and R. Lotan, J. Biol. Chem. 273:35008-35015, 1998). |
| | | The nucleotide sequence of SEQ ID NO:1 is a cDNA sequence and |
| | | comprises a polypeptide encoding sequence (nucleotide 1 to 1209) |
| | | encoding a polypeptide of 403 amino acids, the polypeptide of |
| | | SEQ ID NO:30. The nucleotide sequence encoding the |
| | | polypeptide of SEQ ID NO:30 may be identical to the polypeptide |
| | | encoding sequence contained in SEQ ID NO:29 or it may be a |
| | | sequence other than the one contained in SEQ ID NO:29, which, as |
| | | a result of the redundancy (degeneracy) of the genetic code, also |
| | | encodes the polypeptide of SEQ ID NO:30. The polypeptide of the |
| | | SEQ ID NO:30 is structurally related to other proteins of this |
| | | family, having homology and/or structural similarity with Human |
| | | putative G-protein coupled receptor RAIG1 (Y. Cheng and R. |
| | | Lotan, J. Biol. Chem. 273:35008-35015, 1998). |
| 31 | 32 | The nucleotide sequence of SEQ ID NO:31 and the peptide |
| | | sequence encoded thereby (SEQ ID NO:32) are derived from EST |
| | | (Expressed Sequence Tag) sequences. |
| | | Polynucleotides of this invention may be obtained, using |
| | | standard cloning and screening techniques (Sambrook et al., |
| | | Molecular Cloning: A Laboratory Manual, 2^{nd} Ed., Cold Spring |
| | | Harbor Laboratory Press, Cold Spring Harbor, N.Y.(1989)), from a |
| | | cDNA library derived from human brain. |
| | | The gene of this invention maps to human chromosome |
| | | 16p12. |
| 33 | 34 | Gene Name: AXOR15 |
| | | The nucleotide sequence of SEQ ID NO:33 is a cDNA |
| | | sequence and comprises a polypeptide encoding sequence |
| | | (nucleotide 1 to 1260) encoding a polypeptide of 419 amino acids, |
| | | the polypeptide of SEQ ID NO:34. The nucleotide sequence |
| | | encoding the polypeptide of SEQ ID NO:34 may be identical to the |
| | | polypeptide encoding sequence contained in SEQ ID NO:33 or it |
| | | may be a sequence other than the one contained in SEQ ID NO:33, |
| | | which, as a result of the redundancy (degeneracy) of the genetic |
| | | code, also encodes the polypeptide of SEQ ID NO:34. The |
| | | polypeptide of SEQ ID NO:34 is structurally related to other |
| | | proteins of the G-protein Coupled family, having homology and/or |
| | | structural similarity with High affinity Lysophosphatic acid |
| | | receptor [Z. Guo, et. al. Proc. National Acad. Sci. U.S.A 93 |
| | | (1996)]. |
| 35 | 36 | The nucleotide sequence of SEQ ID NO:35 is a cDNA |
| | | sequence and comprises a polypeptide encoding sequence |
| | | (nucleotide 227 to 1334) encoding a polypeptide of 369 amino |
| | | acids, the polypeptide of SEQ ID NO:36. The polypeptide of SEQ |
| | | ID NO:36 is structurally related to other proteins of the G-protein |
| | | Coupled family, having homology and/or structural similarity with |
| | | High affinity Lysophosphatic acid receptor [Z. Guo, et. al. Proc. |
| | | National Acad. Sci. U.S.A 93 (1996)]. |
| | | Polynucleotides of this invention may be obtained, using |
| | | standard cloning and screening techniques, from a cDNA library |
| | | derived from mRNA in cells of humans, using the expressed |
| | | sequence tag (EST) analysis. |
| | | The gene of this invention maps to human chromosome 6. |
| 37 | 38 | Gene Name: HNFJD15 |
| | | HNFJD15 of the invention is structurally related to other |
| | | proteins of the G-Protein Coupled Receptor, as shown by the |
| | | results of sequencing the cDNA encoding human HNFJD15. The |
| | | cDNA sequence contains an open reading frame encoding a |
| | | protein of 396 amino acid residues with a deduced molecular |
| | | weight of 43.71 kDa. HNFJD15 of FIG. 1 (SEQ ID NO:2) has |
| | | about 26.8% identity (using FASTA) in 224 amino acid residues |
| | | with Human Histamine H2 receptor (Neuroreport, 7 (7), 1293- |
| | | 1296 (1996), Accession # X98133). Furthermore, HNFJD15 |
| | | (SEQ ID NO:2) is 23.1% identical to Serotonin 5HT2 receptor |
| | | over 195 amino acid residues (Pro. Natl. Acad. Sci. U.S.A. 92 |
| | | (12), 5441-5445 (1995), Accession # X81835). Furthermore, |
| | | HNFJD15 is 29.6% identical to Dopamine receptor like protein |
| | | D215 over 196 amino acid residues (Genomics, 25 (2), 436-446 |
| | | (1995), Accession # X80175). HNFJD15 gene of FIG. 1 (SEQ |
| | | ID NO:1) has about 57.00% identity (using BLAST) in 212 |
| | | nucleotide residues with Human Neuropeptide Y4 receptor (Bard |
| | | J. A. et al., J Biol. Chem. 270 (45), 26762-26765, (1995), |
| | | Accession # U35232). Furthermore, HNFJD15 (SEQ ID NO:1) |
| | | is 57.08% identical to Human Pancreatic polypeptide receptor |
| | | over 212 nucleotide base residues (Larhammar D. et al., I. Biol. |
| | | Chem. 270 (49), 29123-29128, (1995), Accession # Z66526). |
| | | One polynucleotide of the present invention encoding |
| | | HNFJD15 may be obtained using standard cloning and screening, |
| | | from a cDNA library derived from mRNA in cells of human |
| | | neutrophil (Activated), human fetal brain and human leukocytes |
| | | using the expressed sequence tag (EST) analysis. |
| 39 | 40 | Gene Name: HEOAD54 |
| | | HEOAD54 of the invention is structurally related to other |
| | | proteins of the G-protein coupled receptor family, as shown by the |
| | | results of sequencing the cDNA encoding human HEOAD54. The |
| | | cDNA sequence of SEQ ID NO:1 contains an open reading frame |
| | | (nucleotide number 236 to 1504) encoding a polypeptide of 423 |
| | | amino acids of SEQ ID NO:2. The amino acid sequence of Table 1 |
| | | (SEQ ID NO:2) has about 40.2% identity (using FASTA) in 291 |
| | | amino acid residues with human probable G-protein coupled |
| | | receptor, HM74 (Accession # P49019, Nomura, H. et al, Int. |
| | | Immunol. 5: 1239-1249, 1993). Furthennore, HEOAD54 (SEQ ID |
| | | NO:2) is 27.3% identical to human P2U purinoceptor over 341 |
| | | amino acid residues (Accession # P41231, Parr, C. E. et al, Proc. |
| | | Natl. Sci. U.S.A. 91: 3275-3279, 1994). The nucleotide sequence |
| | | of Table 1 (SEQ ID NO:1) has about 98.25% identity (using |
| | | BLAST) in 171 nucleotide residues with yc63g05.rl Homo |
| | | sapiens cDNA clone 85400 5′ (Accession # T72122, Wilson, R. et |
| | | al, WashU-Merck EST project, Unpublished, 1995). Furthermore, |
| | | HEOAD54 (SEQ ID NO:1) is 55.59% identical to human G- |
| | | protein coupled receptor mRNA over 349 nucleic acid residues |
| | | (Accession # U35398, An, S. et al, Unpublished, 1995). |
| | | One polynucleotide of the present invention encoding |
| | | HEOAD54 may be obtained using standard cloning and screening, |
| | | from a cDNA library derived from mRNA in cells of human |
| | | Eosinophils using the expressed sequence tag EST analysis. |

Preferred polypeptides and polynucleotides of the present invention are expected to have, inter alia, similar biological functions/properties to their homologous polypeptides and polynucleotides. Furthermore, preferred polypeptides and polynucleotides of the present invention have at least one biological gene activity.

The present invention also relates to partial or other polynucleotide and polypeptide sequences which were first identified prior to the determination of the corresponding full length sequences, described above in Table 1. Specifically, SEQ ID NOS: 3, 7, 17, 18, 27, 31, and 35 are partial polynucleotide sequences and SEQ ID NOS: 4, 8, 28, 32 and 36 are partial polypeptide sequences.

Accordingly, in a further aspect, the present invention provides for an isolated polynucleotide comprising:

(a) a nucleotide sequence which has at least 70% identity, preferably at least 80% identity, more preferably at least 90% identity, yet more preferably at least 95% identity, even more preferably at least 97-99% identity to the partial sequence over the entire length of the partial sequence;

(b) a nucleotide sequence which has at least 70% identity, preferably at least 80% identity, more preferably at least 90% identity, yet more preferably at least 95% identity, even more preferably at least 97-99% identity, to the partial sequence set forth above over the entire length of the partial sequence;

(c) the polynucleotide partial sequence; or

(d) a nucleotide sequence encoding a polypeptide which has at least 70% identity, preferably at least 80% identity, more preferably at least 90% identity, yet more preferably at least 95% identity, even more preferably at least 97-99% identity, to the corresponding partial amino acid sequence set forth above over the entire length of the partial amino acid sequence.

The present invention further provides for a polypeptide which:

(a) comprises an amino acid sequence which has at least 70% identity, preferably at least 80% identity, more preferably at least 90% identity, yet more preferably at least 95% identity, most preferably at least 97-99% identity, to that of the partial amino acid sequence set forth above over the entire length of the partial amino acid sequence;

(b) has an amino acid sequence which is at least 70% identity, preferably at least 80% identity, more preferably at least 90% identity, yet more preferably at least 95% identity, most preferably at least 97-99% identity, to the partial amino acid sequence over the entire length of the partial amino acid sequence;

(c) comprises the partial amino acid sequence; and

(d) is the polypeptide of the partial amino acid sequence;

as well as polypeptides encoded by a polynucleotide comprising the partial polynucleotide sequence.

Polynucleotides of the invention can also be obtained from natural sources such as genomic DNA libraries or can be synthesized using well known and commercially available techniques.

When polynucleotides of the present invention are used for the recombinant production of polypeptides of the present invention, the polynucleotide may include the coding sequence for the mature polypeptide, by itself; or the coding sequence for the mature polypeptide in reading frame with other coding sequences, such as those encoding a leader or secretory sequence, a pre-, or pro- or prepro- protein sequence, or other fusion peptide portions. For example, a marker sequence which facilitates purification of the fused polypeptide can be encoded. In certain preferred embodiments of this aspect of the invention, the marker sequence is a hexa-histidine peptide, as provided in the pQE vector (Qiagen, Inc.) and described in Gentz et al., Proc Natl Acad Sci USA (1989) 86:821-824, or is an HA tag. The polynucleotide may also contain non-coding 5′ and 3′ sequences, such as transcribed, non-translated sequences, splicing and polyadenylation signals, ribosome binding sites and sequences that stabilize mRNA.

Further embodiments of the present invention include polynucleotides encoding corresponding polypeptide variants which comprise the amino acid sequences set forth in SEQ ID NOS: 2, 4, 6, 8, 10, 12, 14, 16, 20, 22, 24, 26, 28, 30, 32, 34 and 36, and in which several, for instance from 5 to 10, 1 to 5, 1 to 3, 1 to 2 or 1, amino acid residues are substituted, deleted or added, in any combination.

Polynucleotides which are identical or sufficiently identical to a nucleotide sequence set forth in Table 1 may be used as hybridization probes for cDNA and genomic DNA or as primers for a nucleic acid amplification (PCR) reaction, to isolate full-length cDNAs and genomic clones encoding polypeptides of the present invention and to isolate cDNA and genomic clones of other genes (including genes encoding homologs and orthologs from species other than human) that have a high sequence similarity to the polynucleotides set forth in Table 1. Typically these nucleotide sequences are 70% identical, preferably 80% identical, more preferably 90% identical, most preferably 95% identical to that of the referent. The probes or primers will generally comprise at least 15 nucleotides, preferably, at least 30 nucleotides and may have at least 50 nucleotides. Particularly preferred probes will have between 30 and 50 nucleotides.

A polynucleotide encoding a polypeptide of the present invention, including homologs and orthologs from species other than human, may be obtained by a process which comprises the steps of screening an appropriate library under stringent hybridization conditions with a labeled probe having the sequence of a sequence set forth in Table 1 or a fragment thereof; and isolating full-length cDNA and genomic clones containing said polynucleotide sequence. Such hybridization techniques are well known to the skilled artisan. Preferred stringent hybridization conditions include overnight incubation at 42° C. in a solution comprising: 50% formamide, 5×SSC (150 mM NaCl, 15 mM trisodium citrate), 50 mM sodium phosphate (pH7.6), 5×Denhardt's solution, 10% dextran sulfate, and 20 microgram/ml denatured, sheared salmon sperm DNA; followed by washing the filters in 0.1×SSC at about 65° C. Thus the present invention also includes polynucleotides obtainable by screening an appropriate library under stringent hybridization conditions with a labeled probe having the sequence of a sequence set forth in Table 1, or a fragment thereof.

The skilled artisan will appreciate that, in many cases, an isolated cDNA sequence will be incomplete, in that the region coding for the polypeptide is cut short at the 5′ end of the cDNA. This is a consequence of reverse transcriptase, an enzyme with inherently low ‘processivity’ (a measure of the ability of the enzyme to remain attached to the template during the polymerization reaction), failing to complete a DNA copy of the mRNA template during 1st strand cDNA synthesis.

There are several methods available and well known to those skilled in the art to obtain full-length cDNAs, or extend short cDNAs, for example those based on the method of Rapid Amplification of cDNA ends (RACE) (see, for example, Frohman et al., PNAS USA 85, 8998-9002, 1988). Recent modifications of the technique, exemplified by the Marathon™ technology (Clontech Laboratories Inc.) for example, have significantly simplified the search for longer cDNAs. In the Marathon™ technology, cDNAs have been prepared from mRNA extracted from a chosen tissue and an ‘adaptor’ sequence ligated onto each end. Nucleic acid amplification (PCR) is then carried out to amplify the ‘missing’ 5′ end of the cDNA using a combination of gene specific and adaptor specific oligonucleotide primers. The PCR reaction is then repeated using ‘nested’ primers, that is, primers designed to anneal within the amplified product (typically an adaptor specific primer that anneals further 3′ in the adaptor sequence and a gene specific primer that anneals further 5′ in the known gene sequence). The products of this reaction can then be analyzed by DNA sequencing and a full-length cDNA constructed either by joining the product directly to the existing cDNA to give a complete sequence, or carrying out a separate full-length PCR using the new sequence information for the design of the 5′ primer.

Recombinant polypeptides of the present invention may be prepared by processes well known in the art from genetically engineered host cells comprising expression systems. Accordingly, in a further aspect, the present invention relates to expression systems or vectors which comprise a polynucleotide or polynucleotides of the present invention, to host cells which are genetically engineered with such expression systems or vectors and to the production of polypeptides of the invention by recombinant techniques. Cell-free translation systems can also be employed to produce such proteins using RNAs derived from the DNA constructs of the present invention.

For recombinant production, host cells can be genetically engineered to incorporate expression systems or portions thereof for polynucleotides of the present invention. Introduction of polynucleotides into host cells can be effected by methods described in many standard laboratory manuals, such as Davis et al., Basic Methods in Molecular Biology (1986) and Sambrook et al., Molecular Cloning: A Laboratory Manual, 2nd Ed., Cold Spring Harbor Laboratory Press, Cold Spring Harbor, N.Y. (1989). Preferred such methods include, for instance, calcium phosphate transfection, DEAE-dextran mediated transfection, transvection, microinjection, cationic lipid-mediated transfection, electroporation, transduction, scrape loading, ballistic introduction or infection.

Representative examples of appropriate hosts include bacterial cells, such as streptococci, staphylococci, E. coli, Streptomyces and Bacillus subtilis cells; fungal cells, such as yeast cells and Aspergillus cells; insect cells such as Drosophila S2 and Spodoptera Sf9 cells; animal cells such as CHO, COS, HeLa, C127, 3T3, BHK, HEK 293 and Bowes melanoma cells; and plant cells.

A great variety of expression systems can be used, for instance, chromosomal, episomal and virus-derived systems, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids. The expression systems may contain control regions that regulate as well as engender expression. Generally, any system or vector which is able to maintain, propagate or express a polynucleotide to produce a polypeptide in a host may be used. The appropriate nucleotide sequence may be inserted into an expression system by any of a variety of well-known and routine techniques, such as, for example, those set forth in Sambrook et al., MOLECULAR CLONING, A LABORATORY MANUAL (supra). Appropriate secretion signals may be incorporated into the desired polypeptide to allow secretion of the translated protein into the lumen of the endoplasmic reticulum, the periplasmic space or the extracellular environment. These signals may be endogenous to the polypeptide or they may be heterologous signals.

If a polypeptide of the present invention is to be expressed for use in screening assays, it is generally preferred that the polypeptide be produced at the surface of the cell. In this event, the cells may be harvested prior to use in the screening assay. If the polypeptide is secreted into the medium, the medium can be recovered in order to recover and purify the polypeptide. If produced intracellularly, the cells must first be lysed before the polypeptide is recovered.

Polypeptides of the present invention can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography is employed for purification. Well-known techniques for refolding proteins may be employed to regenerate active conformation when the polypeptide is denatured during isolation and or purification.

This invention also relates to the use of polynucleotides of the present invention as diagnostic reagents. Detection of a mutated form of the gene characterized by a polynucleotide set forth in Table 1 which is associated with a dysfunction will provide a diagnostic tool that can add to, or define, a diagnosis of a disease, or susceptibility to a disease, which results from under-expression, over-expression or altered expression of the gene. Individuals carrying mutations in the gene may be detected at the DNA level by a variety of techniques.

Nucleic acids for diagnosis may be obtained from a subject's cells, such as from blood, urine, saliva, tissue biopsy or autopsy material. The genomic DNA may be used directly for detection or may be amplified enzymatically by using PCR or other amplification techniques prior to analysis. RNA or cDNA may also be used in similar fashion. Deletions and insertions can be detected by a change in size of the amplified product in comparison to the normal genotype. Point mutations can be identified by hybridizing amplified DNA to labeled nucleotide sequences. Perfectly matched sequences can be distinguished from mismatched duplexes by RNase digestion or by differences in melting temperatures. DNA sequence differences may also be detected by alterations in electrophoretic mobility of DNA fragments in gels, with or without denaturing agents, or by direct DNA sequencing (e.g., Myers et al., Science (1985) 230:1242). Sequence changes at specific locations may also be revealed by nuclease protection assays, such as RNase and S1 protection or the chemical cleavage method (see Cotton et al., Proc Natl Acad Sci USA (1985) 85: 4397-4401). In another embodiment, an array of oligonucleotides probes comprising nucleotide sequence or fragments thereof can be constructed to conduct efficient screening of e.g., genetic mutations. Array technology methods are well known and have general applicability and can be used to address a variety of questions in molecular genetics including gene expression, genetic linkage, and genetic variability (see for example: M. Chee et al., Science, Vol 274, pp 610-613 (1996)).

The diagnostic assays offer a process for diagnosing or determining a susceptibility to the Diseases through detection of mutation in the gene by the methods described. In addition, such diseases may be diagnosed by methods comprising determining from a sample derived from a subject an abnormally decreased or increased level of polypeptide or mRNA. Decreased or increased expression can be measured at the RNA level using any of the methods well known in the art for the quantitation of polynucleotides, such as, for example, nucleic acid amplification, for instance PCR, RT-PCR, RNase protection, Northern blotting and other hybridization methods. Assay techniques that can be used to determine levels of a protein, such as a polypeptide of the present invention, in a sample derived from a host are well-known to those of skill in the art. Such assay methods include radioimmunoassays, competitive-binding assays, Western Blot analysis and ELISA assays.

Thus in another aspect, the present invention relates to a diagnostic kit which comprises:

(a) a polynucleotide of the present invention, preferably the nucleotide sequence set forth in Table 1, or a fragment thereof;

(b) a nucleotide sequence complementary to that of (a);

(c) a polypeptide of the present invention, preferably the polypeptide set forth in Table 1, or a fragment thereof; or

(d) an antibody to a polypeptide of the present invention, preferably to the polypeptide set forth in Table 1.

It will be appreciated that in any such kit, (a), (b), (c) or (d) may comprise a substantial component. Such a kit will be of use in diagnosing a disease or susceptibility to a disease, particularly infections such as bacterial, fungal, protozoan and viral infections, particularly infections caused by HIV-1 or HIV-2; pain; cancers; diabetes, obesity; anorexia; bulimia; asthma; Parkinson's disease; acute heart failure; hypotension; hypertension; urinary retention; osteoporosis; angina pectoris; myocardial infarction; stroke; ulcers; asthma; allergies; benign prostatic hypertrophy; migraine; vomiting; psychotic and neurological disorders, including anxiety, schizophrenia, manic depression, depression, delirium, dementia, and severe mental retardation; and dyskinesias, such as Huntington's disease or Gilles dela Tourett's syndrome, amongst others.

The nucleotide sequences of the present invention are also valuable for chromosome identification. The sequence is specifically targeted to, and can hybridize with, a particular location on an individual human chromosome. The mapping of relevant sequences to chromosomes according to the present invention is an important first step in correlating those sequences with gene associated disease. Once a sequence has been mapped to a precise chromosomal location, the physical position of the sequence on the chromosome can be correlated with genetic map data. Such data are found in, for example, V. McKusick, Mendelian Inheritance in Man (available on-line through Johns Hopkins University Welch Medical Library). The relationship between genes and diseases that have been mapped to the same chromosomal region are then identified through linkage analysis (coinheritance of physically adjacent genes).

The differences in the cDNA or genomic sequence between affected and unaffected individuals can also be determined. If a mutation is observed in some or all of the affected individuals but not in any normal individuals, then the mutation is likely to be the causative agent of the disease.

The polypeptides of the invention or their fragments or analogs thereof, or cells expressing them, can also be used as immunogens to produce antibodies immunospecific for polypeptides of the present invention. The term “immunospecific” means that the antibodies have substantially greater affinity for the polypeptides of the invention than their affinity for other related polypeptides in the prior art.

Antibodies generated against polypeptides of the present invention may be obtained by administering the polypeptides or epitope-bearing fragments, analogs or cells to an animal, preferably a non-human animal, using routine protocols. For preparation of monoclonal antibodies, any technique which provides antibodies produced by continuous cell line cultures can be used. Examples include the hybridoma technique (Kohler, G. and Milstein, C., Nature (1975) 256:495-497), the trioma technique, the human B-cell hybridoma technique (Kozbor et al., Immunology Today (1983) 4:72) and the EBV-hybridoma technique (Cole et al., MONOCLONAL ANTIBODIES AND CANCER THERAPY, pp. 77-96, Alan R. Liss, Inc., 1985).

Techniques for the production of single chain antibodies, such as those described in U.S. Pat. No. 4,946,778, can also be adapted to produce single chain antibodies to polypeptides of this invention. Also, transgenic mice, or other organisms, including other mammals, may be used to express humanized antibodies.

The above-described antibodies may be employed to isolate or to identify clones expressing the polypeptide or to purify the polypeptides by affinity chromatography.

Antibodies against polypeptides of the present invention may also be employed to treat the Diseases, amongst others.

In a further aspect, the present invention relates to genetically engineered soluble fusion proteins comprising a polypeptide of the present invention, or a fragment thereof, and various portions of the constant regions of heavy or light chains of immunoglobulins of various subclasses (IgG, IgM, IgA, IgE). Preferred as an immunoglobulin is the constant part of the heavy chain of human IgG, particularly IgG1, where fusion takes place at the hinge region. In a particular embodiment, the Fc part can be removed simply by incorporation of a cleavage sequence which can be cleaved with blood clotting factor Xa. Furthermore, this invention relates to processes for the preparation of these fusion proteins by genetic engineering, and to the use thereof for drug screening, diagnosis and therapy. A further aspect of the invention also relates to polynucleotides encoding such fusion proteins. Examples of fusion protein technology can be found in International Patent Application Nos. WO94/29458 and WO94/22914.

Another aspect of the invention relates to a method for inducing an immunological response in a mammal which comprises inoculating the mammal with a polypeptide of the present invention, adequate to produce antibody and/or T cell immune response to protect said animal from the Diseases hereinbefore mentioned, amongst others. Yet another aspect of the invention relates to a method of inducing immunological response in a mammal which comprises, delivering a polypeptide of the present invention via a vector directing expression of the polynucleotide and coding for the polypeptide in vivo in order to induce such an immunological response to produce antibody to protect said animal from diseases.

A further aspect of the invention relates to an immunological/vaccine formulation (composition) which, when introduced into a mammalian host, induces an immunological response in that mammal to a polypeptide of the present invention wherein the composition comprises a polypeptide or polynucleotide of the present invention. The vaccine formulation may further comprise a suitable carrier. Since a polypeptide may be broken down in the stomach, it is preferably administered parenterally (for instance, subcutaneous, intramuscular, intravenous, or intradermal injection). Formulations suitable for parenteral administration include aqueous and non-aqueous sterile injection solutions which may contain anti-oxidants, buffers, bacteriostats and solutes which render the formulation instonic with the blood of the recipient; and aqueous and non-aqueous sterile suspensions which may include suspending agents or thickening agents. The formulations may be presented in unit-dose or multi-dose containers, for example, sealed ampoules and vials and may be stored in a freeze-dried condition requiring only the addition of the sterile liquid carrier immediately prior to use. The vaccine formulation may also include adjuvant systems for enhancing the immunogenicity of the formulation, such as oil-in water systems and other systems known in the art. The dosage will depend on the specific activity of the vaccine and can be readily determined by routine experimentation.

Polypeptides of the present invention are responsible for many biological functions, including many disease states, in particular one or more of the Diseases hereinbefore mentioned. It is therefore desirous to devise screening methods to identify compounds which stimulate or which inhibit the function of the polypeptide. Accordingly, in a further aspect, the present invention provides for a method of screening compounds to identify those which stimulate or which inhibit the function of the polypeptide. In general, agonists or antagonists may be employed for therapeutic and prophylactic purposes for such Diseases as hereinbefore mentioned. Compounds may be identified from a variety of sources, for example, cells, cell-free preparations, chemical libraries, and natural product mixtures. Such agonists, antagonists or inhibitors so-identified may be natural or modified substrates, ligands, receptors, enzymes, etc., as the case may be, of the polypeptide; or may be structural or functional mimetics thereof (see Coligan et al., Current Protocols in Immunology 1(2):Chapter 5 (1991)).

The screening method may simply measure the binding of a candidate compound to the polypeptide, or to cells or membranes bearing the polypeptide, or a fusion protein thereof by means of a label directly or indirectly associated with the candidate compound. Alternatively, the screening method may involve competition with a labeled competitor. Further, these screening methods may test whether the candidate compound results in a signal generated by activation or inhibition of the polypeptide, using detection systems appropriate to the cells bearing the polypeptide. Inhibitors of activation are generally assayed in the presence of a known agonist and the effect on activation by the agonist by the presence of the candidate compound is observed. Constitutively active polypeptides may be employed in screening methods for inverse agonists or inhibitors, in the absence of an agonist or inhibitor, by testing whether the candidate compound results in inhibition of activation of the polypeptide. Further, the screening methods may simply comprise the steps of mixing a candidate compound with a solution containing a polypeptide of the present invention, to form a mixture, measuring activity in the mixture, and comparing the activity of the mixture to a standard. Fusion proteins, such as those made from Fc portion and polypeptide, as hereinbefore described, can also be used for high-throughput screening assays to identify antagonists for the polypeptide of the present invention (see D. Bennett et al., J Mol Recognition, 8:52-58 (1995); and K. Johanson et al., J Biol Chem, 270(16):9459-9471 (1995)).

One screening technique includes the use of cells which express the receptor of this invention (for example, transfected CHO cells) in a system which measures extracellular pH or intracellular calcium changes caused by receptor activation. In this technique, compounds may be contacted with cells expressing the receptor polypeptide of the present invention. A second messenger response, e.g., signal transduction, pH changes, or changes in calcium level, is then measured to determine whether the potential compound activates or inhibits the receptor.

Another method involves screening for receptor inhibitors by determining inhibition or stimulation of receptor-mediated cAMP and/or adenylate cyclase accumulation. Such a method involves transfecting a eukaryotic cell with the receptor of this invention to express the receptor on the cell surface. The cell is then exposed to potential antagonists in the presence of the receptor of this invention. The amount of cAMP accumulation is then measured. If the potential antagonist binds the receptor, and thus inhibits receptor binding, the levels of receptor-mediated cAMP, or adenylate cyclase, activity will be reduced or increased.

Another method for detecting agonists or antagonists for the receptor of the present invention is the yeast based technology as described in U.S. Pat. No. 5,482,835.

The polynucleotides, polypeptides and antibodies to the polypeptide of the present invention may also be used to configure screening methods for detecting the effect of added compounds on the production of mRNA and polypeptide in cells. For example, an ELISA assay may be constructed for measuring secreted or cell associated levels of polypeptide using monoclonal and polyclonal antibodies by standard methods known in the art. This can be used to discover agents which may inhibit or enhance the production of polypeptide (also called antagonist or agonist, respectively) from suitably manipulated cells or tissues.

The polypeptide may be used to identify membrane bound or soluble receptors, if any, through standard receptor binding techniques known in the art. These include, but are not limited to, ligand binding and crosslinking assays in which the polypeptide is labeled with a radioactive isotope (for instance, ¹²⁵I), chemically modified (for instance, biotinylated), or fused to a peptide sequence suitable for detection or purification, and incubated with a source of the putative receptor (cells, cell membranes, cell supernatants, tissue extracts, bodily fluids). Other methods include biophysical techniques such as surface plasmon resonance and spectroscopy. These screening methods may also be used to identify agonists and antagonists of the polypeptide which compete with the binding of the polypeptide to its receptors, if any. Standard methods for conducting such assays are well understood in the art.

Examples of potential polypeptide antagonists include antibodies or, in some cases, oligonucleotides or proteins which are closely related to the ligands, substrates, receptors, enzymes, etc., as the case may be, of the polypeptide, e.g., a fragment of the ligands, substrates, receptors, enzymes, etc.; or small molecules which bind to the polypeptide of the present invention but do not elicit a response, so that the activity of the polypeptide is prevented.

Thus, in another aspect, the present invention relates to a screening kit for identifying agonists, antagonists, ligands, receptors, substrates, enzymes, etc. for polypeptides of the present invention; or compounds which decrease or enhance the production of such polypeptides, which comprises:

(a) a polypeptide of the present invention;

(b) a recombinant cell expressing a polypeptide of the present invention;

(c) a cell membrane expressing a polypeptide of the present invention; or

(d) antibody to a polypeptide of the present invention;

which polypeptide is set forth in Table 1.

It will be appreciated that in any such kit, (a), (b), (c) or (d) may comprise a substantial component.

It will be readily appreciated by the skilled artisan that a polypeptide of the present invention may also be used in a method for the structure-based design of an agonist, antagonist or inhibitor of the polypeptide, by:

(a) determining in the first instance the three-dimensional structure of the polypeptide;

(b) deducing the three-dimensional structure for the likely reactive or binding site(s) of an agonist, antagonist or inhibitor;

(c) synthesizing candidate compounds that are predicted to bind to or react with the deduced binding or reactive site; and

(d) testing whether the candidate compounds are indeed agonists, antagonists or inhibitors.

It will be further appreciated that this will normally be an iterative process.

In a further aspect, the present invention provides methods of treating abnormal conditions such as, for instance, infections such as bacterial, fungal, protozoan and viral infections, particularly infections caused by HIV-1 or HIV-2; pain; cancers; diabetes, obesity; anorexia; bulimia; asthma; Parkinson's disease; acute heart failure; hypotension; hypertension; urinary retention; osteoporosis; angina pectoris; myocardial infarction; stroke; ulcers; asthma; allergies; benign prostatic hypertrophy; migraine; vomiting; psychotic and neurological disorders, including anxiety, schizophrenia, manic depression, depression, delirium, dementia, and severe mental retardation; and dyskinesias, such as Huntington's disease or Gilles dela Tourett's syndrome, related to either an excess of, or an under-expression of polypeptide activity.

If the activity of the polypeptide is in excess, several approaches are available. One approach comprises administering to a subject in need thereof an inhibitor compound (antagonist) as hereinabove described, optionally in combination with a pharmaceutically acceptable carrier, in an amount effective to inhibit the function of the polypeptide, such as, for example, by blocking the binding of ligands, substrates, receptors, enzymes, etc., or by inhibiting a second signal, and thereby alleviating the abnormal condition. In another approach, soluble forms of the polypeptides still capable of binding the ligand, substrate, enzymes, receptors, etc. in competition with endogenous polypeptide may be administered. Typical examples of such competitors include fragments of the polypeptide.

In still another approach, expression of the gene encoding endogenous polypeptide can be inhibited using expression blocking techniques. Known such techniques involve the use of antisense sequences, either internally generated or separately administered (see, for example, O'Connor, J Neurochem (1991) 56:560 in Oligodeoxynucleotides as Antisense Inhibitors of Gene Expression, CRC Press, Boca Raton, Fla. (1988)). Alternatively, oligonucleotides which form triple helices with the gene can be supplied (see, for example, Lee et al., Nucleic Acids Res (1979) 6:3073; Cooney et al., Science (1988) 241:456; Dervan et al., Science (1991) 251:1360). These oligomers can be administered per se or the relevant oligomers can be expressed in vivo.

For treating abnormal conditions related to an under-expression of a polypeptide of the present invention and its activity, several approaches are also available. One approach comprises administering to a subject a therapeutically effective amount of a compound which activates a polypeptide of the present invention, i.e., an agonist as described above, in combination with a pharmaceutically acceptable carrier, to thereby alleviate the abnormal condition. Alternatively, gene therapy may be employed to effect the endogenous production of the polypeptide and polynucleotide by the relevant cells in the subject. For example, a polynucleotide of the invention may be engineered for expression in a replication defective retroviral vector, as discussed above. The retroviral expression construct may then be isolated and introduced into a packaging cell transduced with a retroviral plasmid vector containing RNA encoding a polypeptide of the present invention such that the packaging cell now produces infectious viral particles containing the gene of interest. These producer cells may be administered to a subject for engineering cells in vivo and expression of the polypeptide in vivo. For an overview of gene therapy, see Chapter 20, Gene Therapy and other Molecular Genetic-based Therapeutic Approaches, (and references cited therein) in Human Molecular Genetics, T Strachan and A P Read, BIOS Scientific Publishers Ltd (1996). Another approach is to administer a therapeutic amount of a polypeptide of the present invention in combination with a suitable pharmaceutical carrier.

In a further aspect, the present invention provides for pharmaceutical compositions comprising a therapeutically effective amount of a polypeptide, such as the soluble form of a polypeptide of the present invention, agonist/antagonist peptide or small molecule compound, in combination with a pharmaceutically acceptable carrier or excipient. Such carriers include, but are not limited to, saline, buffered saline, dextrose, water, glycerol, ethanol, and combinations thereof. The invention further relates to pharmaceutical packs and kits comprising one or more containers filled with one or more of the ingredients of the aforementioned compositions of the invention. Polypeptides and other compounds of the present invention may be employed alone or in conjunction with other compounds, such as therapeutic compounds.

The composition will be adapted to the route of administration, for instance by a systemic or an oral route. Preferred forms of systemic administration include injection, typically by intravenous injection. Other injection routes, such as subcutaneous, intramuscular, or intraperitoneal, can be used. Alternative means for systemic administration include transmucosal and transdermal administration using penetrants such as bile salts or fusidic acids or other detergents. In addition, if a polypeptide or other compounds of the present invention can be formulated in an enteric or an encapsulated formulation, oral administration may also be possible. Administration of these compounds may also be topical and/or localized, in the form of salves, pastes, gels, and the like.

The dosage range required depends on the choice of peptide or other compounds of the present invention, the route of administration, the nature of the formulation, the nature of the subject's condition, and the judgment of the attending practitioner. Suitable dosages, however, are in the range of 0.1-100 μg/kg of subject. Wide variations in the needed dosage, however, are to be expected in view of the variety of compounds available and the differing efficiencies of various routes of administration. For example, oral administration would be expected to require higher dosages than administration by intravenous injection. Variations in these dosage levels can be adjusted using standard empirical routines for optimization, as is well understood in the art.

Polypeptides used in treatment can also be generated endogenously in the subject, in treatment modalities often referred to as “gene therapy” as described above. Thus, for example, cells from a subject may be engineered with a polynucleotide, such as a DNA or RNA, to encode a polypeptide ex vivo, and for example, by the use of a retroviral plasmid vector. The cells are then introduced into the subject.

Polynucleotide and polypeptide sequences form a valuable information resource with which to identify further sequences of similar homology. This is most easily facilitated by storing the sequence in a computer readable medium and then using the stored data to search a sequence database using well known searching tools, such as GCC. Accordingly, in a further aspect, the present invention provides for a computer readable medium having stored thereon a polynucleotide comprising a sequence set forth in Table 1 and/or a polypeptide sequence encoded thereby.

The following definitions are provided to facilitate understanding of certain terms used frequently hereinbefore.

“Antibodies” as used herein includes polyclonal and monoclonal antibodies, chimeric, single chain, and humanized antibodies, as well as Fab fragments, including the products of an Fab or other immunoglobulin expression library.

“Isolated” means altered “by the hand of man” from the natural state. If an “isolated” composition or substance occurs in nature, it has been changed or removed from its original environment, or both. For example, a polynucleotide or a polypeptide naturally present in a living animal is not “isolated,” but the same polynucleotide or polypeptide separated from the coexisting materials of its natural state is “isolated”, as the term is employed herein.

“Polynucleotide” generally refers to any polyribonucleotide or polydeoxribonucleotide, which may be unmodified RNA or DNA or modified RNA or DNA. “Polynucleotides” include, without limitation, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, “polynucleotide” refers to triple-stranded regions comprising RNA or DNA or both RNA and DNA. The term “polynucleotide” also includes DNAs or RNAs containing one or more modified bases and DNAs or RNAs with backbones modified for stability or for other reasons. “Modified” bases include, for example, tritylated bases and unusual bases such as inosine. A variety of modifications may be made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short polynucleotides, often referred to as oligonucleotides.

“Polypeptide” refers to any peptide or protein comprising two or more amino acids joined to each other by peptide bonds or modified peptide bonds, i.e., peptide isosteres. “Polypeptide” refers to both short chains, commonly referred to as peptides, oligopeptides or oligomers, and to longer chains, generally referred to as proteins. Polypeptides may contain amino acids other than the 20 gene-encoded amino acids. “Polypeptides” include amino acid sequences modified either by natural processes, such as post-translational processing, or by chemical modification techniques which are well known in the art. Such modifications are well described in basic texts and in more detailed monographs, as well as in a voluminous research literature. Modifications may occur anywhere in a polypeptide, including the peptide backbone, the amino acid side-chains and the amino or carboxyl termini. It will be appreciated that the same type of modification may be present to the same or varying degrees at several sites in a given polypeptide. Also, a given polypeptide may contain many types of modifications. Polypeptides may be branched as a result of ubiquitination, and they may be cyclic, with or without branching. Cyclic, branched and branched cyclic polypeptides may result from post-translation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination (see, for instance, PROTEINS—STRUCTURE AND MOLECULAR PROPERTIES, 2nd Ed., T. E. Creighton, W. H. Freeman and Company, New York, 1993; Wold, F., Post-translational Protein Modifications: Perspectives and Prospects, pgs. 1-12 in POSTTRANSLATIONAL COVALENT MODIFICATION OF PROTEINS, B. C. Johnson, Ed., Academic Press, New York, 1983; Seifter et al., “Analysis for protein modifications and nonprotein cofactors”, Meth Enzymol (1990) 182:626-646 and Rattan et al., “Protein Synthesis: Post-translational Modifications and Aging”, Ann NY Acad Sci (1992) 663:48-62).

“Variant” refers to a polynucleotide or polypeptide that differs from a reference polynucleotide or polypeptide, but retains essential properties. A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence, as discussed below. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more substitutions, additions, deletions in any combination. A substituted or inserted amino acid residue may or may not be one encoded by the genetic code. A variant of a polynucleotide or polypeptide may be a naturally occurring such as an allelic variant, or it may be a variant that is not known to occur naturally. Non-naturally occurring variants of polynucleotides and polypeptides may be made by mutagenesis techniques or by direct synthesis.

“Identity,” as known in the art, is a relationship between two or more polypeptide sequences or two or more polynucleotide sequences, as the case may be, as determined by comparing the sequences. In the art, “identity” also means the degree of sequence relatedness between polypeptide or polynucleotide sequences, as the case may be, as determined by the match between strings of such sequences. “Identity” can be readily calculated by known methods, including but not limited to those described in (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., SIAM J Applied Math., 48: 1073 (1988). Methods to determine identity are designed to give the largest match between the sequences tested. Moreover, methods to determine identity are codified in publicly available computer programs. Computer program methods to determine identity between two sequences include, but are not limited to, the GCG program package (Devereux, J., et al., Nucleic Acids Research 12(1): 387 (1984)), BLASTP, BLASTN, and FASTA (Atschul, S. F. et al., J. Molec. Biol. 215: 403-410 (1990). The BLAST X program is publicly available from NCBI and other sources (BLAST Manual, Altschul, S., et al., NCBI NLM NIH Bethesda, Md. 20894; Altschul, S., et al., J. Mol. Biol. 215: 403-410 (1990). The well known Smith Waterman algorithm may also be used to determine identity.

Parameters for polypeptide sequence comparison include the following:

1) Algorithm: Needleman and Wunsch, J. Mol Biol. 48: 443-453 (1970)

Comparison matrix: BLOSSUM62 from Hentikoff and Hentikoff, Proc. Natl. Acad. Sci. USA. 89:10915-10919 (1992)

Gap Penalty: 12

Gap Length Penalty: 4

A program useful with these parameters is publicly available as the “gap” program from Genetics Computer Group, Madison Wis. The aforementioned parameters are the default parameters for peptide comparisons (along with no penalty for end gaps).

Parameters for polynucleotide comparison include the following:

1) Algorithm: Needleman and Wunsch, J. Mol Biol. 48: 443-453 (1970)

Comparison matrix: matches=+10, mismatch=0

Gap Penalty: 50

Gap Length Penalty: 3

Available as: The “gap” program from Genetics Computer Group, Madison Wis. These are the default parameters for nucleic acid comparisons.

A preferred meaning for “identity” for polynucleotides and polypeptides, as the case may be, are provided in (1) and (2) below.

(1) Polynucleotide embodiments further include an isolated polynucleotide comprising a polynucleotide sequence having at least a 50, 60, 70, 80, 85, 90, 95, 97 or 100% identity to the reference polynucleotide sequence, wherein said polynucleotide sequence may be identical to the reference sequence set forth in Table 1 or may include up to a certain integer number of nucleotide alterations as compared to the reference sequence, wherein said alterations are selected from the group consisting of at least one nucleotide deletion, substitution, including transition and transversion, or insertion, and wherein said alterations may occur at the 5′ or 3′ terminal positions of the reference nucleotide sequence or anywhere between those terminal positions, interspersed either individually among the nucleotides in the reference sequence or in one or more contiguous groups within the reference sequence, and wherein said number of nucleotide alterations is determined by multiplying the total number of nucleotides in the sequence by the integer defining the percent identity divided by 100 and then subtracting that product from said total number of nucleotides in the sequence, or:

nₙ≦xₙ−(xₙ·y),

wherein nₙ is the number of nucleotide alterations, xₙ is the total number of nucleotides in the sequence, y is 0.50 for 50%, 0.60 for 60%, 0.70 for 70%, 0.80 for 80%, 0.85 for 85%, 0.90 for 90%, 0.95 for 95%, 0.97 for 97% or 1.00 for 100%, and · is the symbol for the multiplication operator, and wherein any non-integer product of xₙ and y is rounded down to the nearest integer prior to subtracting it from xₙ. Alterations of a polynucleotide sequence encoding the polypeptide sequence may create nonsense, missense or frameshift mutations in this coding sequence and thereby alter the polypeptide encoded by the polynucleotide following such alterations.

By way of example, a polynucleotide sequence of the present invention may be identical to the reference sequence set forth in Table 1, that is it may be 100% identical, or it may include up to a certain integer number of amino acid alterations as compared to the reference sequence such that the percent identity is less than 100% identity. Such alterations are selected from the group consisting of at least one nucleic acid deletion, substitution, including transition and transversion, or insertion, and wherein said alterations may occur at the 5′ or 3′ terminal positions of the reference polynucleotide sequence or anywhere between those terminal positions, interspersed either individually among the nucleic acids in the reference sequence or in one or more contiguous groups within the reference sequence. The number of nucleic acid alterations for a given percent identity is determined by multiplying the total number of amino acids in polypeptide sequence the integer defining the percent identity divided by 100 and then subtracting that product from said total number of amino acids in the sequence set forth in Table 1, or:

nₙ≦xₙ−(xₙ·y),

wherein nₙ is the number of amino acid alterations, xₙ is the total number of amino acids in the amino acid sequence, y is, for instance 0.70 for 70%, 0.80 for 80%, 0.85 for 85% etc., · is the symbol for the multiplication operator, and wherein any non-integer product of xₙ and y is rounded down to the nearest integer prior to subtracting it from xₙ.

(2) Polypeptide embodiments further include an isolated polypeptide comprising a polypeptide having at least a 50, 60, 70, 80, 85, 90, 95, 97 or 100% identity to a polypeptide reference sequence, wherein the polypeptide sequence may be identical to the reference sequence or may include up to a certain integer number of amino acid alterations as compared to the reference sequence, wherein said alterations are selected from the group consisting of at least one amino acid deletion, substitution, including conservative and non-conservative substitution, or insertion, and wherein said alterations may occur at the amino- or carboxy-terminal positions of the reference polypeptide sequence or anywhere between those terminal positions, interspersed either individually among the amino acids in the reference sequence or in one or more contiguous groups within the reference sequence, and wherein said number of amino acid alterations is determined by multiplying the total number of amino acids in the sequence by the integer defining the percent identity divided by 100 and then subtracting that product from said total number of amino acids in the sequence selected from Table 1, or:

nₐ≦xₐ−(xₐ·y),

wherein nₐ is the number of amino acid alterations, xₐ is the total number of amino acids in the amino acid sequence, y is 0.50 for 50%, 0.60 for 60%, 0.70 for 70%, 0.80 for 80%, 0.85 for 85%, 0.90 for 90%, 0.95 for 95%, 0.97 for 97% or 1.00 for 100%, and · is the symbol for the multiplication operator, and wherein any non-integer product of xₐ and y is rounded down to the nearest integer prior to subtracting it from xₐ.

By way of example, a polypeptide sequence of the present invention may be identical to the reference sequence, that is it may be 100% identical, or it may include up to a certain integer number of amino acid alterations as compared to the reference sequence such that the percent identity is less than 100% identity. Such alterations are selected from the group consisting of at least one amino acid deletion, substitution, including conservative and non-conservative substitution, or insertion, and wherein said alterations may occur at the amino- or carboxy-terminal positions of the reference polypeptide sequence or anywhere between those terminal positions, interspersed either individually among the amino acids in the reference sequence or in one or more contiguous groups within the reference sequence. The number of amino acid alterations for a given % identity is determined by multiplying the total number of amino acids in the reference sequence the integer defining the percent identity divided by 100 and then subtracting that product from said total number of amino acids in the reference sequence, or:

nₐ≦xₐ−(xₐ·y),

wherein nₐ is the number of amino acid alterations, xₐ is the total number of amino acids in, y is, for instance 0.70 for 70%, 0.80 for 80%, 0.85 for 85% etc., and · is the symbol for the multiplication operator, and wherein any non-integer product of xₐ and y is rounded down to the nearest integer prior to subtracting it from xₐ.

“Fusion protein” refers to a protein encoded by two, often unrelated, fused genes or fragments thereof. In one example, EP-A-0 464 discloses fusion proteins comprising various portions of constant region of immunoglobulin molecules together with another human protein or part thereof. In many cases, employing an immunoglobulin Fc region as a part of a fusion protein is advantageous for use in therapy and diagnosis resulting in, for example, improved pharmacokinetic properties [see, e.g., EP-A 0232 262]. On the other hand, for some uses it would be desirable to be able to delete the Fc part after the fusion protein has been expressed, detected and purified.

All publications, including but not limited to patents and patent applications, cited in this specification are herein incorporated by reference as if each individual publication were specifically and individually indicated to be incorporated by reference herein as though fully set forth.

### EXAMPLES

### Example 1

### Mammalian Cell Expression

The receptors of the present invention are expressed in either human embryonic kidney 293 (HEK293) cells or adherent dhfr CHO cells. To maximize receptor expression, typically all 5′ and 3′ untranslated regions (UTRs) are removed from the receptor cDNA prior to insertion into a pCDN or pCDNA3 vector. The cells are transfected with individual receptor cDNAs by lipofectin and selected in the presence of 400 mg/ml G418. After 3 weeks of selection, individual clones are picked and expanded for further analysis. HEK293 or CHO cells transfected with the vector alone serve as negative controls. To isolate cell lines stably expressing the individual receptors, about 24 clones are typically selected and analyzed by Northern blot analysis. Receptor mRNAs are generally detectable in about 50% of the G418-resistant clones analyzed.

### Example 2

### Ligand Bank for Binding and Functional Assays

A bank of over 200 putative receptor ligands has been assembled for screening. The bank comprises: transmitters, hormones and chemokines known to act via a human seven transmembrane (7TM) receptor; naturally occurring compounds which may be putative agonists for a human 7TM receptor, non-mammalian, biologically active peptides for which a mammalian counterpart has not yet been identified; and compounds not found in nature, but which activate 7TM receptors with unknown natural ligands. This bank is used to initially screen the receptor for known ligands, using both functional (i.e. calcium, cAMP, microphysiometer, oocyte electrophysiology, etc, see below) as well as binding assays.

### Example 3

### Ligand Binding Assays

Ligand binding assays provide a direct method for ascertaining receptor pharmacology and are adaptable to a high throughput format. The purified ligand for a receptor is radiolabeled to high specific activity (50-2000 Ci/mmol) for binding studies. A determination is then made that the process of radiolabeling does not diminish the activity of the ligand towards its receptor. Assay conditions for buffers, ions, pH and other modulators such as nucleotides are optimized to establish a workable signal to noise ratio for both membrane and whole cell receptor sources. For these assays, specific receptor binding is defined as total associated radioactivity minus the radioactivity measured in the presence of an excess of unlabeled competing ligand. Where possible, more than one competing ligand is used to define residual nonspecific binding.

### Example 4

### Functional Assay in Xenopus Oocytes

Capped RNA transcripts from linearized plasmid templates encoding the receptor cDNAs of the invention are synthesized in vitro with RNA polymerases in accordance with standard procedures. In vitro transcripts are suspended in water at a final concentration of 0.2 mg/ml. Ovarian lobes are removed from adult female toads, Stage V defolliculated oocytes are obtained, and RNA transcripts (10 ng/oocyte) are injected in a 50 nl bolus using a microinjection apparatus. Two electrode voltage clamps are used to measure the currents from individual Xenopus oocytes in response to agonist exposure. Recordings are made in Ca2+ free Barth's medium at room temperature. The Xenopus system can be used to screen known ligands and tissue/cell extracts for activating ligands.

### Example 5

### Microphysiometric Assays

Activation of a wide variety of secondary messenger systems results in extrusion of small amounts of acid from a cell. The acid formed is largely as a result of the increased metabolic activity required to fuel the intracellular signaling process. The pH changes in the media surrounding the cell are very small but are detectable by the CYTOSENSOR microphysiometer (Molecular Devices Ltd., Menlo Park, Calif.). The CYTOSENSOR is thus capable of detecting the activation of a receptor which is coupled to an energy utilizing intracellular signaling pathway such as the G-protein coupled receptor of the present invention.

### Example 6

### Extract/Cell Supernatant Screening

A large number of mammalian receptors exist for which there remains, as yet, no cognate activating ligand (agonist). Thus, active ligands for these receptors may not be included within the ligand banks as identified to date. Accordingly, the 7TM receptor of the invention is also functionally screened (using calcium, cAMP, microphysiometer, oocyte electrophysiology, etc., functional screens) against tissue extracts to identify natural ligands. Extracts that produce positive functional responses can be sequentially subfractionated until an activating ligand is isolated and identified.

### Example 7

### Calcium and cAMP Functional Assays

7TM receptors which are expressed in HEK 293 cells have been shown to be coupled functionally to activation of PLC and calcium mobilization and/or cAMP stimulation or inhibition. Basal calcium levels in the HEK 293 cells in receptor-transfected or vector control cells were observed to be in the normal, 100 nM to 200 nM, range. HEK 293 cells expressing recombinant receptors are loaded with fura 2 and in a single day>150 selected ligands or tissue/cell extracts are evaluated for agonist induced calcium mobilization. Similarly, HEK 293 cells expressing recombinant receptors are evaluated for the stimulation or inhibition of cAMP production using standard cAMP quantitation assays. Agonists presenting a calcium transient or cAMP fluctuation are tested in vector control cells to determine if the response is unique to the transfected cells expressing receptor.

What is claimed is:

1. An isolated polynucleotide comprising the nucleotide sequence set forth in SEQ ID NO:1.

2. The isolated polynucleotide of claim 1 consisting of the nucleotide sequence set forth in SEQ ID NO:1.

3. An isolated polynucleotide that encodes a polypeptide comprising the amino acid sequence set forth in SEQ ID NO:2.

4. The isolated polynucleotide of claim 3 wherein the polynucleotide encodes the amino acid sequence set forth in SEQ ID NO:2.

5. An isolated polypeptide comprising the amino acid sequence set forth in SEQ ID NO:2.

6. The isolated polypeptide of claim 5 consisting of the amino acid sequence set forth in SEQ ID NO:2.

7. An expression vector comprising the isolated polynucleotide of claim 3 when said expression vector is present in a compatible host cell.

8. An isolated host cell comprising the expression vector of claim 7.

9. A process for producing a Mucilage polypeptide comprising culturing the host cell of claim 8 and recovering the polypeptide from the culture.

10. A membrane of the host cell of claim 9 expressing said polypeptide.
